(19) United States
(12) Patent Application Publication
(10) Pub. No.: US 2026/0007402 A1
(43) Pub. Date: Jan. 8, 2026

(54) MEDICAL THREAD ASSEMBLY FOR CANNULA INSERTION

(71) Applicant: MHmedi. Co., Ltd., Seoul (KR)

(72) Inventor: Hwa Su KIM, Seoul (KR)

(73) Assignee: MHmedi. Co., Ltd., Seoul (KR)

(21) Appl. No.: 19/240,293

(22) Filed: Jun. 17, 2025

(30) Foreign Application Priority Data
Jul. 7, 2024 (KR) 20-2024-0001213

(51) Int. Cl. A61B 17/06

(52) U.S. Cl. CPC A61B 17/06166; A61B 2017/06176

(57) ABSTRACT

Disclosed is a medical thread for cannula insertion including: a thorn area portion arranged at a central area in a lengthwise direction and formed on a surface thereof with a thorn; a first non-thorn area portion extending from an one end in a lengthwise direction of the thorn area portion and excluding a thorn formed on a surface thereof; and a second non-thorn area portion extending from an opposite end in the lengthwise direction of the thorn area portion and excluding a thorn formed on a surface thereof.

### CROSS-REFERENCE TO RELATED APPLICATIONS

The present application claims the benefit of priority to Korean Utility Model Application No. 20-2024-0001213, filed on Jul. 7, 2024. The disclosure of the above-listed application is hereby incorporated by reference herein in their entirety.

### BACKGROUND OF THE INVENTION

### 1. Field of the Invention

The present invention relates to a plastic surgery tool, and more particularly, to a medical thread assembly for cannula insertion to insert a medical thread into a cannula.

### 2. Description of the Related Art

Cosmetics and massage have been consistently used to prevent skin aging, and recently, wrinkle improvement procedures have become widely performed.

Among the wrinkle improvement procedures, face lift refers to a scheme that pulls the skin upward to remove the extra skin, and then sews the skin up. Although the above procedure is effective in speed, it is not easy to readily select the procedure because a surgical risk may be high, scars may leave in front of the ears or on a forehead line, and it may take over a month to recover. Further, the aged skin not only continues to have more wrinkles but also becomes thin, thereby bringing the side effect of accelerating an aging process.

In order to compensate for the above shortcomings of the surgical procedures, the so-called lifting procedure has been widely performed, in which a medical thread harmless to the human body is inserted into the skin to stimulate the skin tissue, thereby promoting skin contraction and regeneration, and further, the medical thread conglutinated to the skin tissue is pulled upward and fixed, thereby lifting the sagging skin tissue. The lifting procedure is widely used because it is simpler than conventional surgical procedures and does not interfere with daily life.

The lifting procedure usually uses a lifting procedure tool having a form in which a medical thread is inserted into a cannula. The cannula generally has a length of 8 to 12 cm, and the medical thread has a length of 10 to 15 cm longer than the cannula.

Accordingly, the conventional plastic surgery tools are assumed that a single medical thread is inserted into the skin tissue in one procedure. In this procedure approach, because a puncture procedure is required to proceed whenever a medical thread is inserted, surgical time may be longer and a fatigue of an operator (e.g., plastic surgeon) may be increased.

In addition, because the conventional plastic surgery tools is required to be used only one time, a burden on the procedure cost is not small.

### RELATED ART

Korean patent registration No. 10-2237586 (THREAD INSERTER FOR SKIN LIFTING)

### SUMMARY OF THE INVENTION

In order to solve the above problems in the art, the present invention provides a medical thread for cannula insertion and a medical thread assembly having the same, such that,
first, two or more strands of medical threads are inserted upon one procedure to improve the procedure time and efficiency, and
second, a cannula is reused after disinfection or the like to reduce procedure costs.

In order to achieve the above objects, the medical thread for cannula insertion of the present invention may include a thorn area portion, a first non-thorn area portion, and a second non-thorn area portion.

The thorn area portion is arranged in a central area in a lengthwise direction and formed on a surface thereof with thorns.

The first non-thorn area portion extends from one lengthwise end of the thorn area portion and does not have thorns on a surface thereof.

The second non-thorn area portion extends from an opposite lengthwise end of the thorn area portion and does not have thorns on a surface thereof.

In the medical thread for cannula insertion of the present invention, the thorn area portion may have a length of 16 to 50 cm which is twice or more than a length of the cannula. The first non-thorn area portion may be fixedly coupled to a rear end of an insertion needle and have a length of 1.5 to 2.5 cm, and the second non-thorn area portion may have a length of 0.5 to 2.5 cm.

In the medical thread for cannula insertion of the present invention, the thorn may have an inverted trapezoidal shape having lateral sides slantingly opening to both sides in the lengthwise direction while protruding outward perpendicular to the lengthwise direction.

The medical thread assembly for cannula insertion of the present invention may include the medical thread and the insertion needle described above.

The insertion needle may have a length longer than a length of the cannula and an outer diameter smaller than an inner diameter of the cannula. The insertion needle may have a rear end to fixedly couple an open end of the first non-thorn area portion of the medical thread.

In the medical thread assembly for cannula insertion of the present invention, the insertion needle may be configured as a cylinder communicated therein and having a front end that is closed, and may include a rear end formed therein with a rear coupling portion that is accommodated with the open end of the first non-visual area portion and then pressed and fit at both side in a width direction to fix and couple the open end of the first non-thorn area portion.

In the medical thread assembly for cannula insertion of the present invention, the insertion needle may be configured to have a blunt outer surface at the front end.

In the medical thread assembly for cannula insertion of the present invention, the insertion needle may be inserted through the rear opening of the cannula and then drawn out through the front opening of the cannula. The insertion needle may be cut and removed at the first non-thorn area portion after at least a part of the first non-thorn area portion is drawn out from the front opening.

The medical thread for cannula insertion and the medical thread assembly having the same subject to the above configuration according to the present invention can be manufactured and stored separately to form (manufacture) the length of the medical thread freely, that is, long, so that multiple (two or more) strands of medical thread can be inserted in one procedure, and as a result, the procedure time can be reduced and the procedure efficiency can be remarkably increased.

In addition, The medical thread for cannula insertion and the medical thread assembly having the same according to the present invention can be manufactured and stored separately from the cannula, so that the cannula can be reused through disinfection, and as a result, the procedure cost can be significantly reduced.

### BRIEF DESCRIPTION OF THE DRAWINGS

FIG. 1 is a perspective view showing a medical thread for cannula insertion according to the present invention.

FIG. 2 is a perspective view showing a medical thread assembly according to the present invention.

FIGS. 3A to 3D shows a method of inserting the medical thread assembly according to the present invention into a cannula in use.

### DETAILED DESCRIPTION OF THE INVENTION

Hereinafter, the present invention will be described in detail with reference to the attached drawings.

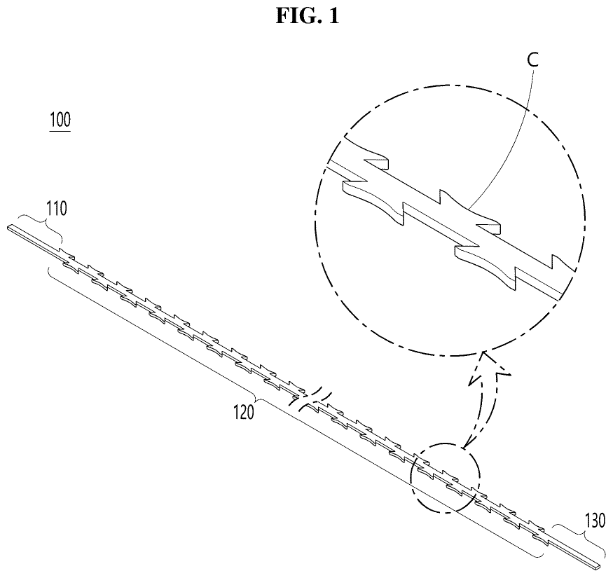

FIG. 1 is a perspective view showing a medical thread for cannula insertion according to the present invention.

As shown in FIG. 1, a medical thread 100 for cannula insertion may be used for purposes such as: promoting skin contraction, skin regeneration and the like after being inserted into the skin tissue to stimulates the skin tissue; and lifting the sagging skin tissue after the medical thread 100 conglutinated to the skin tissue is pulled rearward.

It is preferable that the medical thread 100 be formed of a bioabsorbable material absorbed into the skin after a predetermined period of time. Polylactic acid, polydioxanone, poly-glycolic acid, polycaprolactone, copolymers of lactic acid and glycolic acid, and the like may be used as the bioabsorbable material. However, the medical thread 100 may be formed of a bio-non-absorbable material such as polypropylene, nylon, or a mixture thereof, depending on the embodiment.

The medical thread 100 may include a first non-thorn area portion 110, a thorn area portion 120, and a second non-thorn area portion 130 in a lengthwise direction.

The first non-thorn area portion 110 refers to an area in which no thorns C are formed on an outer surface thereof and may be formed in one end area in the lengthwise direction of the medical thread 100.

The first non-thorn area portion 110 may have an open end coupled and fixed to a rear end of an insertion needle 200 described later.

The first non-thorn area 110 may be temporarily fixed in a form so as to be drawn out from a front opening of a cannula 300 described later, bent rearward, and then put over an edge of the front opening, and may be desirably configured to have a minimum length of 1.5 cm or more in order to achieve the above function/purpose. When the first non-thorn area portion 110 has the length less than 1.5 cm, the first non-thorn area portion 110 may be difficult to be temporarily put across and fixed to the edge of the front opening of the cannula 300 depending on the material of the medical thread 100, and the first non-thorn area portion 110 may be sucked into the front opening of the cannula 300 during a procedure, thereby resulting in procedure failure. Meanwhile, when the first non-thorn area portion 110 has the length more than 2.5 cm, it is not preferable because the area of the medical thread 100 exposed outside the front opening of the cannula 300 may be large during the procedure, thereby, rather, interfering with the procedure, and at length of the thorn area portion 120 for stimulating the skin tissue is reduced.

The thorn area portion 120 refers to an area having an outer surface formed thereon with the thorns C, and may extend from the first non-thorn area portion 110. Considering that a length of one insertion in the thorn area portion 120 is also about 8 to 10 cm when a needle length of the cannula in plastic surgery is generally 8 to 10 cm, the thorn area portion 120 may have the minimum length formed to be at least twice the length of the cannula needle, for example, 16 to 20 cm for consecutive insertions of two or more times. However, because it is realistically difficult to treat insertion of the medical thread 100 consecutively 5 times, and difficulties in manufacturing, storage, and procedure are increased when the length of the medical thread 100 is excessively long, a maximal consecutive insertion may be set as 5 times. In this case, the thorn area portion 120 may have a maximum length of 40 to 50 cm.

In the thorn area portion 120, since the thorn C may perform a function of promoting skin contraction, skin regeneration and the like by stimulating the skin tissue upon being inserted, in addition to a function of lifting the skin tissue by pulling the medical thread conglutinated to the skin tissue rearward, the thorn C may be configured to have a trapezoidal shape protruding in both directions along the length, that is, a shape having a side slope that protrudes outward perpendicular to the lengthwise direction and has lateral sides protruding slantingly on both sides in the lengthwise direction.

In the thorn area portion 120, the thorn C may be formed by cutting and removing a part of an outer side of the medical thread 100 through compression using a compression machine or the like.

In the thorn area portion 120, the thorns C may be arranged at intervals in the lengthwise direction and positioned in a zigzag shape on both sides in a width direction. Accordingly, the thorn area portion 120 may allow the thorns C to strongly and uniformly pull the skin tissue, and also uniformly perform skin tissue stimulation from both directions, so that the effect on plastic surgery can be remarkably increased.

The second non-thorn area portion 130 like the first non-thorn area portion 110, refers to an area in which the thorn C is not formed on an outer surface thereof, and may extend from the thorn area portion 120 at an opposite side of the first non-thorn area portion 110.

The second non-thorn area portion 130 refers to a portion grasped using fingers when the medical thread 100 is inserted into the skin tissue and then pulled rearward, and may be desirably configured to have a minimum length of 0.5 cm or more in order to achieve the above function/purpose. When the second non-thorn area portion 130 has the length less than 0.5 cm, the second non-thorn area portion 130 may not be easily grasped using the fingers. Meanwhile, when the second non-thorn area portion 130 has the length more than 2.5 cm, it may not be preferable because the length of the thorn area portion 120 is reduced without additionally contributing to the function/purpose of the finger grip.

The medical thread 100 may be configured in various sectional shapes such as circular, oval, and rectangular shapes. The medical thread 100 may be formed to have various widths and thicknesses depending on the purpose of procedure. For example, the first and second non-thorn area portions 110 and 130 may be configured with a width of 0.8 to 1.2 mm and a thickness of 0.3 to 0.5 mm, and the thorn area portion 120 may be configured to have the same thickness as the first and second non-thorn area portions (110, 130) and have a width, which is at least 20% wider than the first and second non-thorn area portions 110 and 130, for example, a width of 1.0 to 1.5 mm.

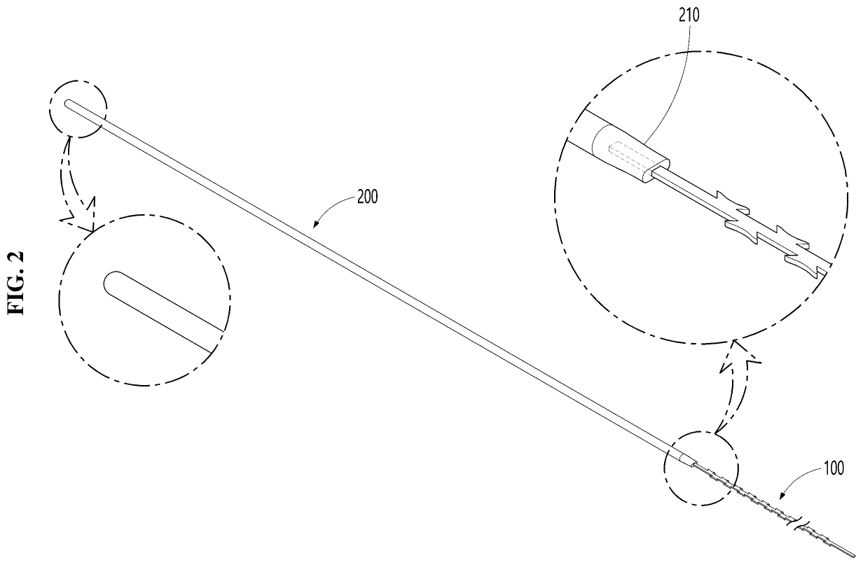

FIG. 2 is a perspective view showing a medical thread assembly according to the present invention.

As shown in FIG. 2, the medical thread assembly according to the present invention may include a medical thread 100 and an insertion needle 200 to insert the medical thread 100 into a cannula 300.

The insertion needle 200 may be provided at a rear end thereof with a rear coupling portion 210 into which a part of the open end of the first non-thorn area portion 110 is internally coupled. The rear coupling portion 210 may have a flat surface shape formed by pressing a rear end of a cylindrical insertion needle 200 symmetrically at both sides in the width direction.

The insertion needle 200 may have an outer diameter smaller than an inner diameter of the cannula 300 so as to be inserted into the cannula 300, and may have a length longer than the cannula 300 so as to be inserted into a rear opening of the cannula 300 and then drawn out from the front opening.

The insertion needle 200 may be configured to have a front end formed in a blunt shape. Accordingly, an operator may be prevented from being injured (e.g., stabbed) during the process of handling the insertion needle 200 (e.g., the insertion needle 200 is inserted into and drawn from the cannula 300).

It is preferable that the insertion needle 200 have a cylindrical shape empty therein, but may also have a cylindrical body shape completely filled therein. In this case, the rear coupling portion 210 may be coupled and fixed to the open end of the first non-thorn area portion 110 using an adhesive or the like.

The insertion needle 200 may be preferably formed of a metal material such as stainless steel or titanium, similar to the cannula 300, but the material is not particularly limited because the insertion needle is not a component inserted into skin tissue, and accordingly, the insertion needle may be formed of plastic or the like.

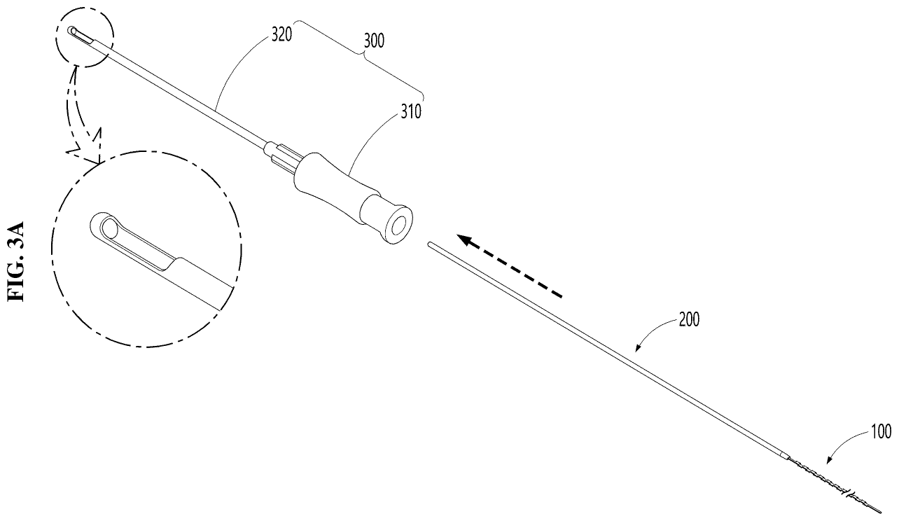

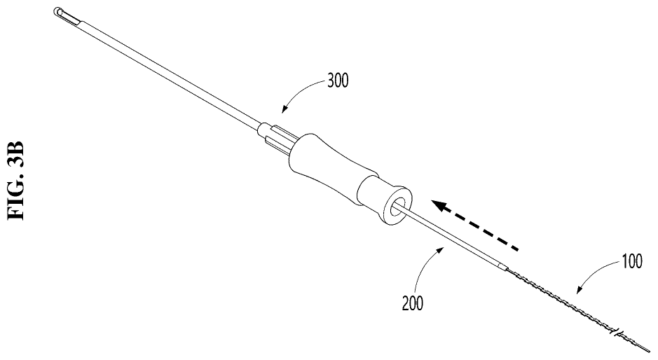

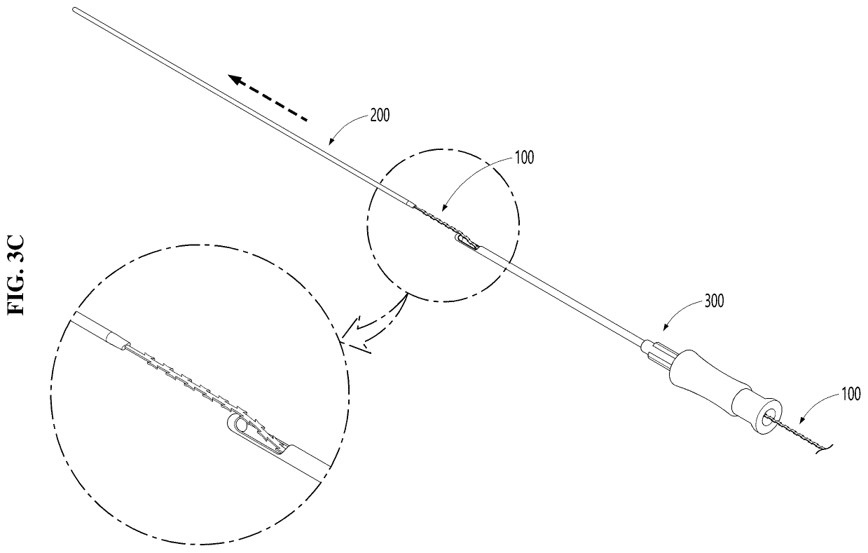

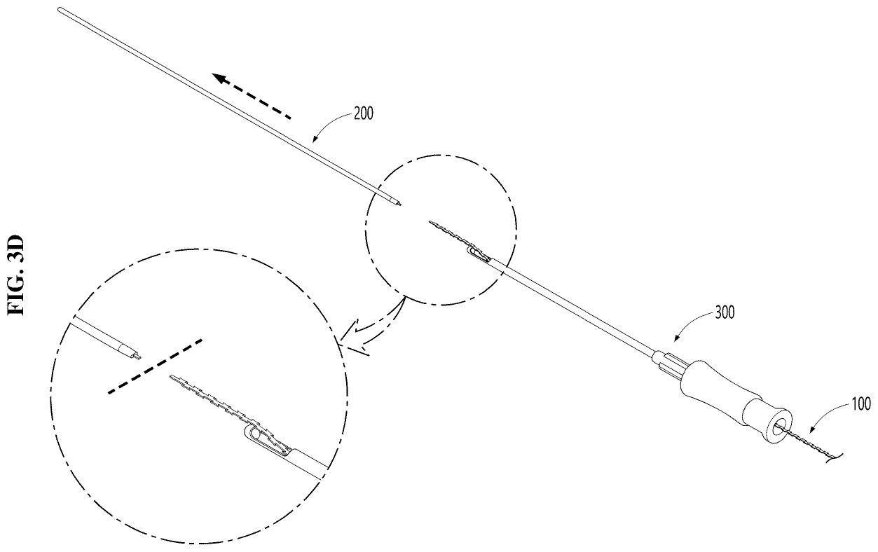

FIGS. 3A to 3D shows a method of inserting the medical thread assembly according to the present invention into a cannula in use.

As shown in FIG. 3A, the medical thread assembly 100 and 200 and the cannula 300 may be prepared.

The cannula 300 is used to insert a medical thread 100 into the skin tissue for cosmetic procedures such as wrinkle improvement and lifting, and may include a cannula hub 310, a cannula needle 320 and the like.

The cannula hub 310 refers to a portion through which the operator grasps the cannula 300 or inserts the medical thread assembly 100 and 200, and may be configured to have a front side coupled to the rear end of the cannula needle 320 and a rear side to be open.

The cannula hub 310 may be formed therein with a flow path communicated in the lengthwise direction. The flow path may be configured to have an inner diameter increasing rearward, thereby allowing the insertion needle 200 to be easily inserted.

The cannula hub 310 may be formed of a plastic material or may be formed of a material partially having elasticity.

The cannula needle 320 is inserted into the skin tissue, and may be formed of a meta harmless to the human body, such as stainless steel and titanium.

The cannula needle 320 may be configured as a cylinder having an inner diameter larger than inner diameters of the medical thread 100 and the insertion needle 200 to allow the medical thread 100 and the insertion needle 200 to be inserted and moved.

The cannula needle 320 may preferably be configured to have a front end sealed and formed in a blunt shape to minimize damage to the skin tissue (e.g., bruising due to blood vessel damage) when being inserted into skin tissue. In this case, the cannula needle 320 may have a front opening arranged at a lateral side at the front end.

The cannula needle 320 may be provided with an inclined guide portion at the sealed front end to guide toward the front opening. The inclined guide portion has a shape inclined forward from a lower end of the inner diameter toward the front end of the front opening. This shape allows the insertion needle 200 to be easily guided and discharged in the direction of the front opening.

The cannula needle 320 is inserted into the skin tissue, and accordingly, may preferably be formed of a metal material harmless to the human body such as stainless steel or titanium.

As shown in FIG. 3B, the insertion needle 200 may be inserted through the rear opening of the cannula 300. The rear opening may be a rear opening of the cannula hub 310.

Since the insertion needle 200 is longer than the cannula needle 320, and when the insertion needle 200 is inserted while the rear end of the insertion needle 200 is held, the front end of the insertion needle 200 may be discharged through the front opening of the cannula 300.

As shown in FIG. 3C, the front end of the insertion needle 200 may be drawn out through the front opening of the cannula 300. At this moment, a part of the first non-thorn area portion 110 coupled to the rear coupling portion of the insertion needle 200 may be drawn out of the front opening. The drawn degree of the first non-thorn area portion 110 may be determined by the operator. For example, when a procedure prevented from irritating the skin tissue is required during inserting the cannula 300 into the skin tissue, only the first non-thorn area portion 110 may be exposed from the front opening. When a procedure for applying stimulation to the skin tissue is required, a part of the thorn area portion 120 may also be exposed. A boundary between the exposed portion and the non-exposed portion may be temporarily fixed in a bent shape put across the rear end of the front opening.

As shown in FIG. 3D, the insertion needle 200 may be cut and separated from the medical thread 100. The cut portion is arranged in the first non-thorn area portion 110. Preferably, the cutting may be performed along a boundary between the rear end of the insertion needle 200 and the first non-thorn area portion 110.

Thereafter, the operator inserts the cannula 300, into which the medical thread 100 is inserted, into the skin tissue. Since the medical thread 100 is longer twice or more than the conventional medical thread, the inserted cannula 300 may be drawn out from the skin tissue only to the skin surface and then arranged in a different direction, so that the cannula 300 may be inserted into the skin tissue one more time. Since the medical thread 100 is inserted into the skin tissue while being exposed to the outside of the cannula 300, the medical thread 100 may be inserted while stimulating the skin tissue through the outer thorns C, thereby increasing the stimulation onto the skin tissue. In addition, the thorns C protrude on both sides in the lengthwise direction, the skin contraction and regeneration effect of stimulating the skin tissue is exhibited in the direction of insertion, and the lifting effect of blocking backward movements is exhibited after the insertion, so that the two effects may be simultaneously achieved by one procedure, thereby maximizing the effectiveness of the procedure.

The present invention has been described as the embodiments, which are intended to illustrate the present invention and a person having ordinary skill in the art may carry out modifications or alterations of the embodiments into other forms. However, since the scope of the rights of the present invention is determined by the claims below, such modifications or alterations may be interpreted as being included in the scope of the rights of the present invention.

What is claimed is:

1. A medical thread assembly for cannula insertion, the medical thread assembly comprising:
a medical thread including:
a thorn area portion having a length greater than twice a length of a cannula, arranged in a central area thereof in a lengthwise direction, and formed on a surface thereof with thorns, in which the thorn is configured to have only an inverted trapezoidal shape having lateral sides slantingly opening to both sides in the lengthwise direction while protruding outward perpendicular to the lengthwise direction;
a first non-thorn area portion extending from one end in the lengthwise direction of the thorn area portion and excluding a thorn formed on a surface thereof; and
a second non-thorn area portion extending from an opposite end in the lengthwise direction of the thorn area portion and excluding a thorn formed on a surface thereof; and
an insertion needle configured as a cylinder communicated therein in the lengthwise direction, having a front end closed in a blunt shape, having a length longer than a length of the cannula and an outer diameter smaller than an inner diameter of the cannula, and having a rear end accommodated in an open end of the first non-visual area portion and then pressed and fit at both side in a width direction to fix and couple the open end of the first non-thorn area portion, wherein
the insertion needle is inserted through a rear opening of the cannula and then drawn out through a front opening of the cannula, and is cut and removed at the first non-thorn area portion after at least a part of the first non-thorn area portion is drawn out from the front opening.

2. The medical thread assembly of claim 1, wherein the thorn area portion has a length of 16 to 50 cm, the first non-thorn area portion has a length of 1.5 to 2.5 cm, and the second non-thorn area portion has a length of 0.5 to 2.5 cm.
